(19) United States
(12) Patent Application Publication
(10) Pub. No.: US 2026/0002113 A1
(43) Pub. Date: Jan. 1, 2026

(54) METHOD FOR PROCESS STATE CLASSIFICATION OF A BIOGAS DIGESTER BASED ON PROCESS VARIABLES OF A BIOGAS PLANT

(71) Applicant: KANADEVIA INOVA AG, Zürich (CH)

(72) Inventors: Bruna AZEVEDO, Allenwinden (CH); Marcel STADELMANN, Luzern (CH); Miriam Estefânia RODRIGUES FERNANDES RABAÇAL, Zurich (CH)

(21) Appl. No.: 19/133,466

(22) PCT Filed: Nov. 28, 2023
(86) PCT No.: PCT/EP2023/083256

§ 371 Date: May 28, 2025

(30) Foreign Application Priority Data
Nov. 29, 2022 (EP) 22210270.9

(51) Int. Cl. C12M 1/36; C12M 1/107; C12M 1/34

(52) U.S. Cl. CPC C12M 41/48; C12M 21/04; C12M 41/34

(57) ABSTRACT

Method for classifying a process state of a biogas digester based on at least one process variable of a biogas plant, the method comprising the steps of: a) Measuring a set of values of at least one process variable of a biogas digester; b) Providing a model that monitors the stability of the biogas digester, wherein the model is trained with a dataset comprising historical data of the digester of the biogas plant, the historical data including the at least one process variable; c) Implementing the measured set of values of the process variable of step a) into the model of step b) with the aid of a data processing unit; d) Identifying whether an outlier occurs in the set of values of step a), after implementing the at least one process variable into the model; e) Classifying a process state indicative of the stability of the biogas digester based on the presence or absence of the identified outlier of step d) with the aid of the data processing unit.

The invention relates to a method for process state classification of a biogas digester based on one or more process variables of a biogas plant.

In recent years, a number of biogas plants have been constructed which successfully contribute to the energy supply. However, in practice, problems with the plant operation are common, which hinders the plants from exploiting their economic potential. One major problem is poor process control of the biogas plants due to inadequate knowledge of the plant operators and under-equipment regarding to online measurement technology, which leads to unfavorably selected substrate feed quantities and substrate compositions, especially in plants in co-substrate operation.

Anaerobic digestions are particularly demanding with respect to process control as the substrate feed is often not homogeneous in terms of space loading and composition. If the digestion substrate is a complex mixed culture, as it is usually the case in biogas production from organic matter (feedstock), this can lead to fluctuations within an adapted bacteria population and thus in the overall metabolic performance.

The worst-case scenario in the operation of biogas plants is the disruption of the digestion process in the digester, which occurs when for example the acid content in the digester reaches a critical value so that adequate methane formation is no longer possible. The digestion process thus comes to a halt. The most common reason for a disruption of the digestion process is an overloading of the digester with organic matter, e.g. due to fluctuations in the feed quantities and substrate compositions. Especially in co-digestions, the risk of process failure is very high.

Control systems have been established to detect a possible future disruption in advance and prevent it by adjusting the conditions in the digester. In many cases, these control systems require the presence of an operator that periodically measures the content of volatile fatty acids (VFA) in the digester. Based on the concentration of VFA and the composition of VFA in a sample from the digester, the operator can determine or “classify” the process state of the digester. Currently these VFA samples are taken manually and are usually analyzed with the aid of high-performance liquid chromatography (HPLC)—which takes 1 to 3 days until the results are available to the operator. In other words, the operator is not “up-to-date” on the actual process state of the digester, because he can only determine the past state of the digester retroactively for the last 1 to 3 days but cannot make any statement about the current state.

There is thus a need for methods or systems that allow providing a statement on the current process state of a biogas plant.

DE 103 544 06 A1 discloses an arrangement for process state classification based on processing of measurement data comprising slow process variables. The arrangement merges the shift in several process variables over a time interval into a process state classification. The disclosed arrangement uses threshold values to classify a process state. However, these threshold values are highly specific for the system to be classified depending on the construction of the system and potential drift of the sensors used for measuring process variables. Hence a “one-fits-all” approach is theoretically possible, but the classification results might not be very representative.

WO 2022/187818 A1 discloses a method to analyze a fermentation process that occurs in a bioreactor. The method uses a trained computing device and one or more machine learning models to analyze the fermentation process and determine a current fermentation state. The disclosed method requires the time-consuming preparation of a large training data set comprising several past disruption events that are labelled by a process expert. Additionally, such a model needs to be re-trained at some point to avoid model degradation, requiring even more labelled training data. To adequately train such a model a sufficiently large amount of past disruption events is required. Since it is difficult to have thousands of past disruption events in the same digester, datasets of different digesters might be combined, which leads to an approach which is not specifically adjusted to a specific digester.

US 2020/074307 A1 discloses a system and a method for performing at least one of assessing, optimizing and/or controlling the performance of an anaerobic digestion plant. The system comprises a user interface, a database for storing inputs, a server that controls the operation of the system and a simulation engine configured to generate a biochemical methane potential, generate estimates of biomethane, electricity and thermal production of the plant.

The problem solved by the present invention is therefore to eliminate the above-described disadvantages of the prior art and providing an improved method for process state classification of a digester based on process variables of a biogas plant.

According to the invention, the problem is solved by a method according to claim 1, a device according to claim 12, a biogas plant according to claim 13, and a model according to claim 14. Preferred embodiments of the invention are given in the dependent claims.

In accordance with the present invention, a method is provided for classifying a process state of a biogas digester based on at least one process variable of a biogas plant. The method comprises the steps of measuring (direct and indirect) a set of values of at least one process variable of a biogas digester (step a)) and the provision of a model that monitors the stability of the biogas digester. This model is trained with a dataset comprising historical data of the digester of the biogas plant, wherein the historical data includes the measured at least one process variable (step b)). In a further step c), the measured set of values of the at least one process variable is implemented into the model with the aid of a data processing unit. In a subsequent step d) the model is used to identify whether an outlier occurs in the implemented set of values of the at least one process variable. Based on the presence or absence of the identified outlier, a process state of the biogas digester that is indicative of the digester's stability is determined with the aid of the data processing unit (step e)).

For the purposes of the invention, the term “set of values” is understood as a series of values measured over a time interval, for example values measured over an hour or a day. This set of values preferably comprises at least two datapoints, each datapoint having first value representing a measurement from the a process variable and a second value representing timepoint of the taken measurement of the process variable, whereby the two datapoints t different timepoints within the time interval.

For the purposes of the invention, the term “process variable” is understood to describe a variable that characterizes the digestion process. Examples for such process variables are the pH, the concentration of specific metabolites, e.g. volatile fatty acids or methane concentration, or the temperature.

For the purposes of the invention, the term “model that monitors the stability” is understood to describe a computer model comprising an algorithm that supervises the stability of the digestion process in the digester.

For the purposes of the invention, the term “dataset comprising historical data” is understood to describe a dataset comprising at least one datapoint, said datapoint including a first value representing a previously taken measurement (a measurement from the past that was taken prior to the current measurement) and a second value representing the timepoint of the previously taken measurement. Preferably the dataset encompasses at least one value representing a multitude of previously taken measurements at different timepoints from the past for one specific process variable. It is clear to the skilled person that the dataset can also comprise a multitude of values from a multitude of timepoints in the past for a multitude of process variables.

For the purposes of the invention, the term “outliers” is used to describe a datapoint in the set of values of the process variable that lies in abnormal distance to other data points in the same dataset or that differs substantially from historical datapoints for a specific process variable. An anomaly could be considered a synonym for outliers in the context of this invention.

For the purpose of better understanding, the method of the invention will be explained in the following with reference to a preferred demonstration example. In this regard, it should be noted that the below disclosed method is a demonstration example and by no means limiting to the method to which the present invention refers. In this preferable demonstration example, the method uses the hydrogen concentration and the methane concentration within the digester as measured process variable for a classification of the process state.

In the first step, the hydrogen concentration [H_{2]} and the methane concentration [CH_{4]} in the digester is measured at several timepoints within the time interval of one day (t₁−tₙ; wherein n>1). It is assumed that elevated concentration of hydrogen in combination with a decreasing concentration of methane compared to respective historical data of the digester is an indication that the digestion reaction in the digestor is not running under ideal conditions and that intermediate products, here hydrogen, are present in a higher-than-preferred concentration. The set of values of the measured hydrogen and methane concentrations within the time interval t₁−tₙ ([H₂] t₁−tₙ) and ([CH₄] t₁−tₙ) are implemented into a preexisting model that monitors the stability of the digester. This model was previously trained with a historical dataset of the digester comprising hydrogen and methane concentrations at various timepoints prior to t₁. When the set of values of the hydrogen and methane concentrations within the time interval t₁−tₙ ([H₂] t₁−tₙ) and ([CH₄] t₁−tₙ) is implemented into the model, the model is used to identify possible outliers in the set of values. In this demonstration example the set of values of the measured hydrogen concentration within time interval t₁−tₙ was found to be higher than expected and the measured methane concentration was found to be lower than expected based on the historical data for these two process variables. These outliers are then detected by the model and subsequently the data processing unit classifies the digester as “unstable” since the outliers in the hydrogen and methane concentration indicate that the digestion reaction in the digestor is not running under ideal conditions.

In the above context, an unstable process is considered as the starting point of an overacidification of the digester, which can occur in case of an intensification of the hydrolysis process or a decreased ability of the microorganisms to degrade the acids due to ammonia inhibition.

It has surprisingly been found that the method according to the invention for the classification of a process state based on the presence or absence of t identified outlier significantly improves the classification accuracy compared to using “filtered” process variables for which outliers have been excluded as described in the prior art. Furthermore, the method according to the invention has the advantage that it is not using absolute values (threshold values)—contrary to the methods described in the prior art.

The implementation of a model that is trained with a dataset comprising historical data of the digester has the advantage that the dataset used for training the model is highly specialized for said digester, which increases the accuracy of the classification. In particular, this highly specialized dataset is more accurate than a “standardized training dataset”—i.e. a single dataset that is used to train multiple models for stability monitoring of different digesters—regarding the specific setup of the digester and its sensors, which avoids a misclassification due a sensor drift. Drift is a natural phenomenon for sensors. It affects basically all sensors regardless and is caused by physical changes in the sensor. The only way to know if a sensor has drifted is through calibration. A typical example of a sensor drift is when a sensor measures a non-changing parameter, e.g. a fixed methane concentration, several times, but the resulting values are not identical, but “drifting”. Another example is a when a sensor measures an in fact changing parameter, but reports identical values.

In a preferred embodiment of the invention the at least one process variable is selected from the group consisting of methane concentration in the biogas, hydrogen concentration in the biogas, ratio of produced biogas over fermented biomass and combinations thereof. Hereby, the term “biogas” refers to the gas which is produced in the biogas digester (also known to the skilled person as “raw biogas”). The concentration of a metabolite in the biogas is measured in a sample from the digester. The term “ratio of produced biogas over fermented biomass” refers to the average biogas flow of the day of process classification (t₁) divided by the average of the mass of biomass fed to the digester—preferably in the past 2 to 14 days, more preferably in the past 5 days, and including day t₁. A preferred unit for the ratio of produced biogas over fermented biomass is Nm³/(h*T), i.e. norm cubic meter (Nm³) per hour (h) per metric ton (t).

For each of the above-mentioned preferred process variables, some preferred outliers detection methods will be described in the following.

In case the at least one process variable is the methane concentration in the biogas, the detection of outliers in step d) of the inventive method preferably involves the use of a bootstrap sampling approach followed by the identification of outliers that are certain numbers of standard deviations away from the predicted value.

If the at least one process variable is the hydrogen concentration in the biogas, the detection of outliers in step d) of the inventive method preferably involves the use of an algorithm that detects “outliers” or “anomalies” in gaussian distributed datasets. A preferred algorithm is the “elliptic_envelope” in Python.

In case the at least one process variable is the hydrogen concentration of biogas another preferred approach will be described in the following:

Firstly, a low pass Butterworth filter is used to reduce noise in the signal of the hydrogen concentration dataset.

Secondly, local peaks in the filtered dataset are detected. As mentioned above, the dataset can be viewed as a series of data points, each with a value for the measured process variable—here the hydrogen concentration in the biogas—and a value for the measurement timepoint. In an orthogonal coordinate system with the time on the X-axis and the process variable, here the hydrogen concentration, on the Y-axis, a function can be assumed that connects all datapoints. In this function the local peaks are assumed to be mathematical minima and maxima, wherein a local minimum is always followed by a local maximum.

In a third step the average absolute vertical distance on the Y-axis (hydrogen concentration) between any two neighboring peaks is calculated and significant peaks are selected. A significant peak is preferably defined as a peak, where the vertical distance to its neighboring peak is above 10-30%, more preferably above 20% of the average vertical distance as calculated above. For all significant peaks, the average vertical distance (μₚ) and the standard deviation (αₚ) are calculated.

In a fourth step a standard peak factor (SPF) is calculated using the following formula (I).

$$SPF = \frac{{μ}_{p} + x \times {α}_{p}}{{μ}_{p}} , \qquad ( I )$$

wherein x is an integer from 1 to 10

In a preferred embodiment of the invention x is an integer from 1 to 4 in formula (I).

In a fifth step the last peak difference (LPD) is calculated by subtracting the hydrogen concentration of the last local peak [H]ₗₚ from the hydrogen concentration of the last data point [H]_{dp} of the dataset using formula (II).

$$L P D = \frac{{[ H ]}_{dp} - {[ H ]}_{lp}}{{μ}_{p}} \qquad ( II )$$

The value of the LPD can be used to identify an increasing or decreasing trend in the process variable—here hydrogen concentration in the biogas. If the value for LPD is negative, it is assumed that the hydrogen concentration is decreasing and therefore a decreasing trend is determined. If the value for LPD is positive, it is assumed that the hydrogen concentration is increasing and therefore an increasing trend for the hydrogen concentration is determined.

In order to evaluate whether a determined trend is significant, the LPD is compared to the SPF. If the absolute value of the LPD (independent of its sign) is higher than the value of the SPF, the last trend (trend originating from the last peak) is considered to be significant. If the last trend is increasing and significant, it is concluded that the hydrogen values are increasing towards outlier values. And this significant increase in hydrogen in combination with lower methane values is sufficient to classify the system as unstable.

In case, the at least one process variable is the ratio of produced biogas over fermented biomass it is preferred to use an algorithm to detect change in its underlying distribution towards lower values using Hoeffding's bounds with moving average-test. A preferred algorithm would be the script “skmultiflow.drift_detection.HDDM_A” written in Python, to detect outliers in step d).

Changes in the underlying distribution towards lower values that occur when the throughput is stable or increasing, the biogas production is not highly oscillating in the past few days, and the biogas production has significantly decreased in the past day, indicate biological instability. Therefore, only the data points of the produced biogas over fermented biomass with detected changes in the underlying distribution towards lower values that additionally fulfil these conditions are identified as “outliers”.

The implementation of one of the above-mentioned preferred process variables or a combination of these variables showed surprisingly reliable results regarding the classification of a process state of a biogas digester. Another advantage of using these process variables is that the method enables an “up-to-date” classification of the process state of the digester, in contrast to the complex and time-consuming measurement of volatile fatty acids (VFA) suggested in the prior art.

In a preferred embodiment, the measured set of values of step a) includes a combination of the concentration of hydrogen in the biogas and the concentration of methane in the biogas as process variables, wherein each process variable is processed separately by the method steps a) to d). Hence, the steps a) to d) are repeated twice, once for the concentration of hydrogen and once for the concentration of methane. Only then, the final step e) is performed and the process state is classified based on the identification of outliers determined in step d) for either one or both process variables. Thereby, the order in which the two process variables are executed is not relevant.

For the above preferred embodiment, preferred ways of classifying the process state of the digester will be described in more detail.
i. If an outlier is detected in the set of values for the methane concentration within a certain time interval, and an outlier is also detected in the set of values for the hydrogen concentration in the same time interval, the process is classified as “unstable”.
ii. If an outlier is only detected in the set of values for the hydrogen concentration within a certain time interval, and no outlier is detected in the set of values for the methane concentration in the same time interval, the process is classified as “stable”.
iii. If an outlier is only detected in the set of values for the methane concentration within a certain time interval and no outlier is detected in the set of values for the hydrogen concentration in the same time interval, the process is classified as “stable”.
iv. If no outliers are detected in both the set of values for the methane concentration and the set of values for the hydrogen concentration within a certain time interval, the process is classified as “stable”.
v. If outliers are detected in both the set of values for the methane concentration and the set of values for the hydrogen concentration, but these outliers do not occur in the same time interval, the process is classified as “stable”.

In the above-described cases i. to v., the time interval is preferably 1 to 30 days, more preferably 21 days and most preferably 5 days.

When the process is classified as unstable, this often indicates that an overacidification of the digester has occurred. An overacidification can be caused by overfeeding the digester or due to ammonia inhibition.

Preferably, f the process state shifts from stable to unstable, an alarm is triggered to alert the operator.

In another preferred embodiment, the measured set of values of step a) includes a combination of the concentration of hydrogen in the biogas and the concentration of methane in the biogas as process variables, wherein a) further involves step measuring the concentration of CO₂ in the biogas as a process measurement verification variable. This process measurement verification variable is measured in accordance with the above-described way of measuring the concentration of hydrogen or methane in the biogas.

Using CO₂ as a process measurement verification variable means that the concentration of CO₂ in the biogas is used to verify the concentration of methane in the biogas. In case the sum of the concentration of CO₂ in the biogas and the concentration of methane in the biogas is less than 99% of the sum of all metabolites in the biogas, it is assumed that the measured concentration of methane is not correct, and therefore the methane concentration cannot be used to classify the digester's state. Therefore, in embodiments in which the concentration of CO₂ is measured, it is preferred that the classification in step e) is not executed or the process is classified as “unclassifiable” if the sum of the concentrations of methane and CO₂ in the biogas is less than 99% of the concentration of all compounds in the biogas.

In a preferred embodiment of the invention the historical data used to train the digester in step b) includes historical data of the digester from the past month, preferably from the past 3 months, more preferably from the past 6 months, even more preferably from the past 9 months, and most preferably from the past 12 months. Hereby, the term “comprising at least historical data from the digester from the previous X months” refers to a dataset which contains values of measured process variables from the past until and including the day of classification.

Preferably, in step b) the model is trained using an algorithm for unsupervised machine learning. The term “unsupervised learning” thereby refers to a machine learning technique that does not require training the model using labeled data. Instead, the model is allowed to work on its own using unlabeled data when discovering information. When the model is trained using unsupervised learning it has the benefit that the model does not rely on labeled data, which avoids a time-consuming data annotation.

In case an outlier was identified, it is preferred that step d) further includes subsequent filtering the set of values of the measured process variable of step a) to detect the root cause of the outlier. This filtering in step d) has the advantage that not every set of values of every measured process variable must be filtered in advance and only the values of a measured variable that produces an outlier is filtered after identification of the latter.

In the above preferred embodiment, steps d) and e) preferably include the following.
If at least one outlier is identified in step d), the measured process variable of step a) is considered to include values that need to be reviewed. This revision involves further processing, specifically filtering, of the measured process variable to find the root cause of the outlier. If a root cause is found, the reviewed values are considered “valid” and in step e) the digester is classified as “not classifiable”. In other words, an identified outlier with a root cause refers to a digester that cannot be classified as “stable” or “unstable” and is therefore “not classifiable”.
On the other hand, if no root cause is found, the reviewed values are considered “invalid” and in step e) the digester is classified as “unstable”. In other words, an identified outlier with no root cause refers to a digester that is “unstable”, since there is no explanation why the outlier occurred.

In an alternative preferred embodiment, each set of values of the measured process variable of step a) is filtered before the implementation into the model in step c) to detect the root cause of a potential outlier. This by default filtering at the stage of step a) has the advantage that only filtered set of values are implemented into the model and the identification of outliers of step d) can be restricted to “valid” values. In this embodiment, the method preferably includes the following:

After measuring the set of values of the at least one process variable in step a), each set of values is labelled as “values to be reviewed”. These values to be reviewed are further processed in a filtering step a2) to identify outliers. If no outlier is found in the set of values to be reviewed, the set of values is implemented into the model in step c).

If at least one outlier is identified, the root cause of this outlier is to be determined. If a root cause is found, the set of values to be reviewed is considered “valid” and the subsequent steps b), c) and d) are skipped and in step e) the digester is classified as “not classifiable”. In other words, an identified outlier with a found root cause refers to a digester that cannot be considered “stable” or “unstable” and has therefore the label “not classifiable”.

If at least one outlier is identified and no root cause can be found, the set of values to be reviewed is considered “invalid” and the subsequent steps b), c) and d) are skipped and in step e), the digester is classified as “unstable”. In other words, an identified outlier with no root cause refers to a digester that is “unstable”, since there is no evidence, why the outlier occurred.

Preferably the step of filtering values to detect the root cause of the outlier uses as a filter criteria a variable selected from the group consisting of agitator torque, feeding slope, and feeding downtime.

Within the context of the invention, the term “agitator torque” refers to the torque of an agitator in the digester, which is used to agitate or mix the biomass inside the digester.

For the purposes of the invention, the term “feeding slope” refers to the amount of biomass that is fed to the digester in a specific time interval. This specific time interval is called “feeding cycle”. Since the biomass is generally fed batch-wise into the digester, the feeding cycle starts, when the feeding starts and ends when the feeding ends. Therefore, the feeding slope refers to the ratio of biomass in [kg] over time in [min] for each cycle. The time between two feeding cycles is called feeding interval.

For the purposes of the invention, the term “feeding downtime” is used to describe the sum of all feeding intervals that exceeds the average feeding interval plus the standard deviation, of all feeding intervals in a day. The feeding downtime represents the time of accumulated unexpected breaks during a day.

It has surprisingly been found that these three variables are highly accurate to determine root causes for outliers identified in the set of values of the at least one process variable.

For example, increased values for the process variable agitator torque can be indicative to a high amount of green waste in the biomass which has a lower methane production yield than food waste. “Green waste” hereby refers to organic waste that originates from plants and can comprise mowed grass, wood, leaves etc., but excludes food products. On the other hand, an increased value in the agitator torque can also indicate that the biomass in the fermenter has a higher density, which could limit the release of produced biogas.

It is further assumed that increased values for the process variable feeding slope may be indicative to a fast-feeding pace, which means that more biomass is fed to the digester than the digester can ferment into biogas. Hence, the biogas quality drops, the relative concentration of methane in the biogas decreases and the relative concentration of byproducts or intermediate products such as H₂ in the biogas increases.

It is further assumed that increased values for the process variable feeding downtime can indicate a longer break between two feeding cycles. Such a prolonged break can decrease the biogas yield over multiple cycles, since in the downtime, no biomass (and thus no substrate that could be converted to biogas) is fed to the fermenter.

For example, a combination of decreased feeding downtime and an increased feeding slope and increase the total amount of biomass feed to the digester. This might lead to a short-term increase in H₂ concentration and decrease in the CH₄ concentration. Therefore, in cases where a significant increase in throughput is detected, deviations in the concentrations of H₂ and CH₄ are not considered to classify the system if the H₂ and CH₄ values quickly return to the regular values. In a preferred embodiment of the invention, the steps a) to d) are repeated twice with different process variables. In the subsequent step e) the classification of the process state of the biogas digester is preferably based on the outliers identified in step d) for all evaluated process variables. It was found that the classification of the process state is more reliable if it is based on two or more process variables that are examined separately.

Preferably the classified process state of step e) is disclosed to an operator. This operator can then manually modify the digestion process based on his knowledge. This combination of the implementation of a trained model and a skilled operator enables an improved biogas production control.

The invention further relates to a device comprising a training data generation unit, a model construction unit and a data processing unit. The training data generation unit is configured to generate training data based on historical data of a digester of a biogas plant. The model construction unit is configured to construct a model by performing learning using the training data. The data processing unit is configured to input into the model a set of values of at least one process variable, to identify outliers in the set of values, and to obtain a classification of a process state of the biogas digester.

The device according to the invention has the advantage that it is not using absolute values (threshold values) contrary to the devices disclosed in the prior art. As described above the implementation of a device including a model that is trained with a dataset comprising historical data of a digester has the advantage, that the dataset for training the model is highly specialized for said digester, which increases the accuracy of the classification. Hence, this device can be implemented into an existing biogas plant to increase the stability of the digester and therefore increase the revenue of the biogas plant.

The invention further relates to a biogas plant comprising a biogas digester to produce biogas, a sensor to measure a set of values of at least one process variable of the biogas digester and a device as described above.

The biogas plant, according to the invention, has the advantage that it is not using absolute values (threshold values) as the biogas plants disclosed in the prior art. The increased stability of the digester results in an increased revenue of the biogas plant.

The invention further relates to a model that monitors the stability of the biogas digester and enables a data processing unit in a biogas plant to perform the method for classifying a process state as described above.

The model according to the invention has the advantage that it increases the stability of the digester, since even minor changes can be detected at an early stage and the operator can. This increased stability of the interfere if necessary. digester results in an increased revenue of the biogas plant.

Further disclosed herein is a method for creating a training data set for classifying a process state of a biogas digester. The method comprises the step of first, measuring a set of values of at least one process variable of a biogas digester and second implement the set of values of the at least one process variable of step a) into an existing or newly created training dataset. These two steps are repeated multiple times for several timepoints to generate a training data set for classifying a process state of a biogas digester.

The above-mentioned method for creating a training dataset has the advantage that the created training dataset can be used to increase the stability of the digester, since slight changes can be detected at an early stage and the operator can interfere if necessary. This increased stability of the digester results in an increased revenue of the biogas plant.

1. Method for classifying a process state of a biogas digester based on at least one process variable of a biogas plant, the method comprising the steps of:
a) Measuring a set of values of at least one process variable of a biogas digester;
b) Providing a model that monitors the stability of the biogas digester, wherein the model is trained with a dataset comprising historical data of the digester of the biogas plant, the historical data including the at least one process variable;
c) Implementing the measured set of values of the process variable of step a) into the model of step b) with the aid of a data processing unit;
d) Identifying whether an outlier occurs in the set of values of step a), after implementing the at least one process variable into the model;
e) Classifying a process state indicative of the stability of the biogas digester based on the presence or absence of the identified outlier of step d) with the aid of the data processing unit.

2. Method according to claim 1, wherein the at least one process variable is selected from the group consisting of methane concentration in the biogas, hydrogen concentration in the biogas, ratio of produced biogas to fermented biomass, and combinations thereof, preferably the least one process variable is the hydrogen concentration in the biogas.

3. Method according to claim 2, wherein the at least one process variable is a combination of the concentration of methane in the biogas and the concentration of hydrogen in the biogas.

4. Method according to claim 3, wherein step a) further includes measuring the concentration of CO₂ in the biogas as a process verification variable.

5. Method according to claim 1, wherein the dataset includes historical data from the digester from the past month, preferably from the past 3 months, more preferably from the past 6 months, even more preferably from the past 9 months, and most preferably from the past 12 months.

6. Method according to claim 1, wherein in step b) the model is trained using an algorithm for unsupervised machine learning.

7. Method according to claim 1, wherein in case an outlier was identified, step d) further includes filtering the set of values of the measured at least one process variable of step a) to detect the root cause of the outlier.

8. Method according to claim 1, wherein the values of the measured at least one process variable are filtered to detect the root cause of a potential outlier prior to implementing the measured set of values of the process variable into the model in step c).

9. Method according to claim 7, wherein the filtering to detect the root cause of the outlier or the potential outlier uses as a filter criterium a variable selected from the group consisting of agitator torque, feeding slope, and feeding downtime.

10. Method according to claim 1, wherein the steps a) to d) are repeated twice with different process variables.

11. Method according to claim 1, wherein the classified process state of step e) is passed on to an operator.

12. A device comprising
a training data generation unit, which generates training data based on historical data of a digester of a biogas plant;
a model construction unit, which constructs a model by performing learning using the training data; and
a data processing unit, which inputs into the model a set of values of at least one process variable, identifies outliers in the set of values, and obtains a classification of a process state indicative of the stability of the biogas digester based on the presence or absence of the identified outlier.

13. A biogas plant comprising:
a biogas digester for the production of biogas;
a sensor for measuring a set of values of at least one process variable of the biogas digester; and
a device according to claim 12.

14. A model that monitors the stability of a biogas digester and operates a data processing unit in a biogas plant to perform the method for classifying a process state according to claim 1.
